(19) United States
(12) Patent Application Publication
(10) Pub. No.: US 2005/0191290 A1
(43) Pub. Date: Sep. 1, 2005

(54) Means and methods for manipulating hypersensitivity-like responses

(76) Inventors: Franciscus Petrus Nijkamp, Houten (NL); Franciscus Antonius Maria Redegeld, Zeist (NL); Aletta Desiree Kraneveld, Nigtevegt (NL); Johannes Gerardus Joseph van de Winkel, Zeist (NL); Gestur Vidarsson, Bilthoven (NL)

Correspondence Address: TRASK BRITT, P.O. BOX 2550, SALT LAKE CITY, UT, 84110, US

(21) Appl. No.: 10/936,433

(22) Filed: Sep. 7, 2004

Related U.S. Application Data
(63) Continuation of application No. PCT/NL03/00167, filed on Mar. 5, 2003.
(60) Provisional application No. 60/362,040, filed on Mar. 6, 2002.

(30) Foreign Application Priority Data
Mar. 6, 2002 (EP) 02075874.4
Jun. 14, 2002 (EP) 02077352.9

(51) Int. Cl. A61K 39/395; A61K 38/10; C12N 5/08

(52) U.S. Cl. 424/131.1; 514/15; 435/372

(57) ABSTRACT

Immunoglobulin light chains (Ig-LC) are produced in excess in animals compared to heavy chains. The present invention implicates Ig-LC in hypersensitivity responses and provides ways for manipulating the responses. The invention further provides a common gamma chain-independent receptor on mast cells capable of mediating the mentioned effects of Ig-LC. In response to activation of the pathway of which the found receptor is a part, a mast cell is activated and stimulated to degranulate.

### CROSS-REFERENCE TO RELATED APPLICATIONS

This application is a continuation of pending PCT International Patent Application No. PCT/NL/03/00167, filed on Mar. 5, 2003, designating the United States of America, and published, in English, as PCT International Publication No. WO 03/074563 A2 on Sep. 12, 2003, and also claims the benefit, under 35 U.S.C. § 119(e) to U.S. Provisional Patent Application Ser. No. 60/362,040, filed on Mar. 6, 2002, the contents of the entirety of both of which are incorporated herein by this reference.

### TECHNICAL FIELD

The invention relates generally to biotechnology and medicine. The invention further relates to the fields of immunology and molecular biology. The invention in particular relates to means and methods for manipulating hypersensitivity responses.

### BACKGROUND

Immunoglobulins (Ig) are important effector molecules of adaptive humoral immune responses. Production of IgE and IgG1 antibodies to innocuous antigens is a reflection of a normal immune response. Binding to Fc receptors, FcεRI or FcγRIII, on mast cells and basophiles, respectively, and subsequent cross-linking of these Fc receptors can trigger hypersensitivity reactions. In patients with allergic syndromes, IgE is thought to play a central role in eliciting immediate hypersensitivity reactions. However, many hypersensitivity diseases do not always correspond with increased serum IgE levels or skin reactivity to common allergens. Studies in epsilon heavy chain gene-targeted mice (IgE-deficient) showed that active anaphylaxis can be induced in the absence of IgE antibodies and was likely due to IgG1. On the other hand, immediate-like hypersensitivity reactions can also be elicited by other antigen-specific factors (1).

Tetrameric Igs are produced and secreted by plasma cells. However, it is well documented that plasma cells also produce and secrete single Ig light chains (Ig-LC) in excess over Ig heavy chains or gamma chains (2-6). In vivo turnover studies in humans demonstrated that apparently only 60% of synthesized Ig-LC was incorporated into isotypic whole Ig and the remaining fraction was released into serum as free Ig light chain (7). Therefore, Ig-LC can be detected at low levels in normal serum and also in urine and in cerebrospinal fluid. Single Ig light chain and Ig light chain dimers bind antigen specifically. Antigen-binding affinities vary and are generally lower, but also affinities similar to or higher than tetrameric Ig were reported (8-15). It is against current dogma that secreted Ig-LC may play a physiological role, although in some instances, free Ig-LC were found to display antigen-specific proteolytic activity (16, 17). However, in this study we show that Ig-LC can transfer immediate hypersensitivity-like responses in mice. These hypersensitivity-like responses are mast cell-mediated and are not detectable in mast cell-deficient mice. Shortly after antigen exposure of Ig-LC-sensitized animals, mast cell activation and local tissue swelling can be detected. We show herein that Ig-LC can serve as a link in the mechanism by which mast cells regulate a number of immune-mediated diseases.

### SUMMARY OF THE INVENTION

Mast cells are important for the development of acute responses in allergic reactions. Thus far, triggering the high-affinity IgE receptor (FcεRI) and the low-affinity IgG-receptor (FcγRIII) are the only routes known to activate mast cells in an antigen-specific manner. The present invention discloses that Ig-LC can exert their action independent from activation of the FcγRIII or FcεRI receptors. Both receptors signal via the common gamma chain and can trigger hypersensitivity reactions via activation of mast cells. Passive sensitization of animals deficient in the common gamma chain (FcRγ−/−) resulted in similar ear swelling responses after hapten challenge as compared to wild-type (C57BL/6) animals (FIG. 1). This result shows that Ig light chains interact with a receptor that does not need the common gamma chain for signaling and thereby excludes FcεRI and FcγRIII or other gamma chain-associated receptors as effector molecules in Ig-LC-induced hypersensitivity.

In one aspect, the present invention thus provides an isolated cell comprising an Ig-LC receptor capable of activating a signal transduction pathway in the cell upon binding and cross-linking of an Ig-LC to the receptor, wherein this signal transduction is independent of the presence of a functional gamma chain on the cell. This isolated cell preferably comprises a mast cell or a gamma chain-associated receptor-deficient cell.

Knowledge of the presence of a gamma chain-independent receptor for Ig-LC opens the route to the identification of the receptor. Many techniques are present in the art to find receptors for ligands. The invention preferably utilizes protein solutions that are enriched for Ig-LC receptors. In one embodiment, the invention, therefore, provides a method for obtaining a protein solution enriched for a receptor for Ig-LC comprising providing a gamma chain-deficient mast cell with Ig-LC and cross-linking the Ig-LC to proteins in its vicinity and purifying Ig-LC and linked proteins from non-linked proteins. This purification can be achieved through many different means. In a preferred embodiment, this is achieved through magnetic beads associated with the Ig-LC. Magnetic beads are coupled to Ig-LC. These beads are then incubated with mast cells or mast cell proteins and after washing the cells, Ig-LC (bait) are chemically cross-linked to cell surface proteins in immediate proximity to the binding site. After lysing the cells, Ig-LC cross-linked cell surface proteins are separated from non-bound proteins using a magnetic device. Proteins are washed and separated using SDS-PAGE. This can be done using gel electrophoresis methods. Proteins can be characterized and identified by sequencing (part of) the proteins from certain positions in the gel. In a preferred embodiment, the receptor comprises a protein with an apparent molecular weight between 37 and 50 kDa and an even more preferred embodiment, the apparent molecular weight is 45 kDa.

Proteins are further characterized and identified with Maldi-TOF mass spectrometry and/or Edman degradation. Binding of Ig-LC-conjugated magnetic beads coupled to mast cells was visualized in the present invention by light microscopy (FIGS. 2B-2D). Binding was specific for light chain, since no binding was detected when beads were conjugated to bovine serum albumin (BSA). The process mentioned above is, of course, very much facilitated by the availability of the results of the genome project. Even very limited information of the sequence can currently be used to at least limit the number of candidate molecules. In many cases, even limited information on the sequence is sufficient to identify a single candidate molecule responsible for the binding to mast cells. Depending on the identified candidates, different methods can be utilized to prove that the molecule is the receptor.

For example, immunofluorescence microscopy has been used to show that FcRn is present intracellularly, as well as on the outside of the plasma membrane of K562 cells. Flow-cytometric analysis shows that K562 cells are able to bind human kappa Ig-LC. The presence of FcRn on the outside of the cell in combination with the ability to bind Ig-LC supports the proposed role of FcRn as the LC-receptor.

Yet another receptor present on mast cells of specific interest for binding Ig-LC is CD63, a transmembrane-4 (TM4) membrane protein. This receptor is expressed by, for example, mast cells, granulocytes and leucocytes and cross-linking of this receptor results in mast cell activation and mediator release.

CD63 is widely expressed on different mammalian cells. In mast cell studies, CD63 has been used as an activation marker, since expression is increased after degranulation. On the other hand, a study in human CD63-transfected rat basophilic cells showed cross-linking of CD63 resulted in mast cell activation and degranulation. Thus far, no ligand for CD63 has been described yet (i.e., orphan receptor).

Thus, in another aspect, the invention provides a purified and/or isolated and/or recombinant Ig-LC receptor like, for example, a CD63 receptor or an FcRn receptor, or a functional part, derivative and/or analogue thereof, capable of activating an Ig-LC-dependent signal transduction pathway in a cell, wherein activation is, for example, but not limited to, an ion channel, activation being independent of the presence of a functional gamma chain receptor on the cell. A functional part of an Ig-LC receptor is a part comprising the same Ig-LC-binding capabilities. A derivative of the mentioned receptor or part thereof, can be obtained through (conservative) amino acid substitution. A functional part, analogue and/or derivative comprises the same Ig-LC-binding capabilities in kind, not necessarily in amount. The functional part may also be incorporated in a proteinaceous molecule capable of binding to Ig-LC. The molecule may be incorporated in the wall of a mast cell or it may be in a soluble form like, for example, many receptors have a cell-bound form and a soluble form. Therefore, the invention also provides a proteinaceous compound capable of binding to Ig-LC and a mast cell, this compound comprising a FcRn receptor or a functional part thereof. The invention also teaches a proteinaceous compound capable of binding to Ig-LC and a mast cell, this compound comprising a CD63 receptor or a functional part thereof. Once a protein is identified, it is within the power of a person skilled in the art to provide a nucleic acid encoding the protein. Thus, the present invention also provides a nucleic acid encoding an Ig-LC receptor of the invention or a functional part, derivative and/or analogue thereof, capable of activating an Ig-LC-dependent signal transduction pathway in a cell. Expression vectors can be made and these can be introduced into target cells. Thus, these vectors are also part of the invention. For more efficient delivery, such vectors may be packaged into gene delivery vehicles such as, for example, virus or virus-like particles. Such virus or virus-like particles are, therefore, also part of the invention. The artisan is further capable of generating antibodies that are specific for the gamma chain-independent Ig-LC receptor.

With the knowledge of the gamma chain-independent Ig-LC receptor, it is possible to find compounds that, at least in part, inhibit the signal transduction pathway of the Ig-LC receptor, an example of such a compound is a molecule that competes with the binding of Ig-LC. This can, for instance, be done through molecular modeling or by high-throughput screening. Moreover, knowledge that Ig-LC is capable of binding to the receptor can be used to find competing molecules. Thus, the invention further provides a compound capable of, at least in part, inhibiting a signal transduction pathway of an Ig-LC receptor of the invention. Preferably, the compound comprises a molecule capable of competing with Ig-LC for binding to a gamma chain-independent Ig-LC receptor. Preferably, the compound comprises an antagonist capable of competing with Ig-LC for binding to a gamma chain-independent Ig-LC receptor. Competing molecules can be tested for their capacity to antagonize the action of an Ig-LC. Preferably, this testing comprises the degranulation of mast cells or an Ig-LC-dependent signal transduction pathway in a cell, more preferably independent of the presence of a functional immunoglobulin or gamma chain-associated receptor on the cell.

In another embodiment, the invention provides a method for determining whether a compound is capable of, at least in part, inhibiting signal transduction of a gamma chain-independent Ig-LC receptor comprising providing a gamma chain receptor-deficient cell comprising the receptor with the compound and determining whether Ig-LC-mediated signal transduction is, at least in part, inhibited in the cell. A suitable antagonist of Ig-LC is an Ig-LC mutated in the receptor binding site such that it is not able to activate the receptor any more. Thus, the invention provides a compound capable of interacting with an Ig-LC receptor capable of preventing the sensitizing of a mast cell. Preferably, this compound comprises an Ig-LC selected for its capacity not to elicit a signal transduction signal.

An antagonist can be used to prevent or reduce the sensitizing of a mast cell. Thus, the invention also provides a method for preventing or reducing the sensitizing of a mast cell comprising providing the mast cell with an Ig-LC antagonist. Preferably, the antagonist comprises a substance capable of binding to an Ig-LC receptor and incapable of binding an antigen or incapable of activating the receptor after binding an antigen.

Now that the receptor is found, it is also possible to manipulate the signal transduction pathway that the receptor is part of. This can be done most easily on the level of the receptor itself. It is, for instance, possible to provide libraries of mutated receptors. These libraries can be used to find a mutant that is capable of activating a mast cell, independent of the presence of a bound Ig-LC/antigen complex. Providing activators or antagonists, or even Ig-LC, one can manipulate activation of a mast cell. Thus, the invention also provides the use of an Ig-LC receptor to modulate a mast cell-activated immune response.

Clinical uses are also within the invention. For instance, an animal suffering or at risk of suffering from a hypersensitivity response can be administered a compound of the invention, thereby reducing the hypersensitivity response or reducing the chance and/or extent with which a hypersensitivity response will appear. In another embodiment, the invention provides a method for reducing a hypersensitivity response in an animal comprising providing the animal with a molecule capable of preventing binding of an Ig-LC to an Ig-LC receptor. The molecule can be a receptor antagonist of the invention. The molecule can also be a molecule capable of binding to an Ig-LC, thereby preventing binding of the bound Ig-LC to a gamma chain-independent receptor or binding of antigen by the receptor-bound Ig-LC. The latter molecule is, for the present invention, called an Ig-LC antagonist or ligand antagonist. The invention further provides an Ig-LC antagonist capable of preventing binding of an Ig-LC to a gamma chain-dependent receptor on mast cells. In one embodiment, a compound capable of, at least in part, inhibiting an Ig-LC signal transduction pathway comprises THP or uromodulin, or a functional part, derivative and/or analogue thereof. In a preferred embodiment, this compound comprises a peptide comprising an amino acid sequence (AHWSGHCCL) (SEQ ID NO:1), or a functional part, derivative, and/or analogue thereof.

For the present invention, a human is also considered to be an animal. In a preferred embodiment, the animal comprises a mammal. More preferably, the mammal comprises a human.

The invention further provides a use of a compound capable of, at least in part, inhibiting an Ig-LC signal transduction pathway, preferably an Ig-LC antagonist or a receptor antagonist, in the preparation of a medicament for the treatment of chronic inflammatory and autoimmune diseases and/or immediate and delayed hypersensitivity responses such as contact dermatitis, asthma, psoriasis, inflammatory bowel disease, rheumatoid arthritis, Sjögren, and systemic lupus erythematosus, and/or multiple sclerosis. Preferably, the medicament is formulated and packaged for parenteral and/or oral administration. Preferably, the compound comprises THP or uromoduline, or a functional part, derivative and/or analogue thereof. In a preferred embodiment, the compound comprises a peptide comprising an amino acid sequence (AHWSGHCCL) (SEQ ID NO:1), or a functional part, derivative, and/or analogue thereof.

The mentioned routes of administration allow the formation of a depot from which a new antagonist is recruited over time, thus allowing for a more prolonged effect of the medicament compared to an intravenous administration. The invention further provides a method of treatment for an animal suffering from, or at risk of suffering from, chronic inflammatory and autoimmune diseases and/or immediate or delayed hypersensitivity-like responses such as asthma, psoriasis, inflammatory bowel disease, rheumatoid arthritis, Sjögren, systemic lupus erythematosus, and/or multiple sclerosis. The method comprises administering to an animal a medicament comprising a compound of the invention such as an Ig-LC antagonist or a gamma chain-independent receptor antagonist and a suitable carrier. In a preferred embodiment, the disease comprises Multiple Sclerosis.

Free Ig-LC are produced and secreted by B lymphocytes and considerable levels of Ig-LC are present in serum. The present invention demonstrates that Ig-LC confer hypersensitivity in naive animals. Mice passively sensitized with trinitrophenol (TNP)- or oxazolone (Ox)-specific Ig-LC develop a cutaneous swelling response, have elevated plasma histamine and show morphologic signs of mast cell degranulation after challenge with relevant antigen. Induction of hypersensitivity is independent of presence of the common gamma chain, excluding a role for Fc- or other gamma chain-associated receptors. Hapten-specific Ig-LC is produced within 24 hours after topical sensitization with low-molecular weight compounds trinitrophenol chloride(2-chloro1,3,5 trinitrobenzene; picryl chloride (PCl)), 2,4-dinitrofluorobenzene (DNFB) or oxazolone by spleen cells from sensitized mice. Although it is clear that IgE and IgG₁ are central to the induction of immediate hypersensitivity reactions, the present invention shows that Ig-LC are similarly able to transfer hypersensitivity to naive animals. Free Ig-LC are an as yet unappreciated factor in the humoral immune response to antigen exposure and can, upon cross-linking, lead to mast cell-dependent immediate hypersensitivity-like reactions.

### DESCRIPTION OF THE FIGURES

FIG. 1: Ear swelling induced by cross-linking of Ig light chains is not dependent on Fc-receptors. Common gamma chain knockouts and control mice (C57B1/6) were passively sensitized with TNP-specific Ig light chain or vehicle (PBS). Ears were challenged with picryl chloride (TNP) and ear thickness was measured two hours later.

FIG. 2A: Quantification of binding of magnetic beads derivatized with albumin (BSA), Ig light chain plus BSA (to improve orientation of Ig light chain). Binding was scored using light microscopy and number of beads attached per cell is shown.

FIG. 2B: Microscopic visualization of binding Ig light chain-coupled magnetic beads to primary cultured murine bone marrow-derived mast cells (BMMC). BMMC incubated with beads coupled to BSA, showing minor degree of binding.

FIG. 2C: Ig light chain-derivatized magnetic beads showing clear rosetting of beads around intact cells.

FIG. 2D: Ig light chain-derivatized magnetic beads (Ig light chain+BSA to correct orientation of the light chains) showing clear rosetting of beads around intact cells.

FIGS. 3A-3C: Expression of CD63 on COS fibroblast-like cells (FIG. 3A), a rat mast cell line RBL-2H3 (FIG. 3B) and primary cultured murine mast cells BMMC (FIG. 3C). Expression is detected by FACS with an antibody AD1 specific for CD63.

FIG. 4: Mast cell dependency of hapten-induced ear swelling in Ig-LC-sensitized mice. Mast cell-deficient (W/W^{v}) or congenic controls (+/+) were intravenously sensitized with TNP-specific Ig light chain (5 mg). Thirty minutes after sensitization, mice were topically challenged with PCl and ear thickness was monitored at two hours after challenge. Ear swelling in W/W^{v} significantly different from +/+, p<0.05. Local reconstitution of the right ear with bone marrow-derived mast cells in W/W^{v} mice, three weeks prior to the experiment resulted in a complete recovery of the sensitivity to Ig light chain.

FIG. 5: Electron micrograph of a mildly degranulated mast cell in the dermis of the ear of a TNP-specific Ig light chain-sensitized mouse at one hour after topical challenge with hapten. Numerous granules remain unaltered, some secretory granules are enlarged, exhibit diminished electron density and release their content (enlargement in b).

FIGS. 6A and 6B: Injection of TNP- or oxazolone-specific Ig light chain resulted in antigen-specific transfer of hapten sensitivity. Mice were intravenously injected with TNP-specific Ig light chain or oxazolone-specific Ig light chain and challenged on the ear with trinitrophenol chloride (PCl) (panel A) or oxazolone (Oxa) (panel B). Controls received vehicle (PBS) and were also topically challenged with both haptens. Ear swelling was measured two hours after challenge.

FIG. 7: Mast cells sensitized with Ig light chain recognize antigen in a specific manner. TNP-specific Ig light chain-sensitized cultured mast cells (BMMC) were incubated with TNP- or oxazolone- (OX-) conjugated SRBC; rosetting cells were scored using light microscopy.

FIG. 8: Antigen-specific Ig light chain is produced after in vivo skin sensitization with low-molecular weight compounds. Immunoblotting of antigen-binding factors specific for 2-chloro-1,3,5-trinitrobenzene (A), dinitrofluorobenzene (B) and oxazolone (C) with Ig kappa light chain-specific antibody.

FIG. 9: Measurement of free kappa Ig light chains in human serum using an Ig kappa light chain-specific ELISA. Samples of six human subjects were analyzed at different dilutions (1000, 2000, and 10,000-fold diluted).

FIG. 10: Intravenous administration of different amounts of F991 at 30 minutes before hapten challenge of Ig light chain-sensitized mice results in a dose-dependent inhibition of ear swelling after hapten challenge. Mice were i.v. sensitized with 2 μg TNP-specific Ig light chain at 30 minutes before ear challenge with TNP. Control mice were injected with PBS instead of Ig light chain.

FIG. 11: Intraperitoneal administration of F991 (50 μg/animal) at four hours or 24 hours before hapten challenge of Ig light chain-sensitized mice completely inhibits induction of ear swelling after hapten challenge. Mice were i.v. sensitized with 2 μg TNP-specific Ig light chain at 30 minutes before ear challenge with TNP. Control mice were injected with PBS instead of Ig light chain.

FIG. 12: Subcutaneous administration of F991 (50 μg/animal) at four hours or 24 hours before hapten challenge of Ig light chain-sensitized mice completely inhibits induction of ear swelling after hapten challenge. Mice were i.v. sensitized with 2 μg TNP-specific Ig light chain at 30 minutes before ear challenge with TNP. Control mice were injected with PBS instead of Ig light chain.

FIG. 13: Topical application of F991 as an ointment on the ears results in dose-dependent inhibition of ear swelling after hapten challenge of Ig light chain-sensitized animals. Application of F991 at 100 μg/g cream corresponds with a local dose of 20 μg per animal.

FIG. 14: Dorsal application (on back of mice) of F991 in ointment does not result in inhibition of ear swelling after hapten challenge of Ig light chain-sensitized animals.

FIG. 15: F991 retains its activity when stored as an ointment at room temperature or at 4° C. for more than three months.

FIG. 16: Topical treatment of DNFB-sensitized mice at four hours before hapten challenge results in complete inhibition of the development of contact sensitivity response as determined by ear swelling at two and 24 hours after challenge.

FIG. 17: Topical treatment of DNFB-sensitized mice after hapten challenge results in decrease of the development of contact sensitivity response as determined by ear swelling four days after challenge.

FIG. 18: Induction of EAE in MOG/pertussis toxin-treated mice is completely suppressed by daily administration of F991 (50 μg/animal i.p.).

FIG. 19: Purification of Ig-LC-binding mast cell membrane proteins. Solubilized mast cell membrane fraction was sequentially incubated with albumin (BSA) end Ig-LC-coated beads as described in Example 2. Isolated proteins were fractionated on SDS-PAGE and visualized by silver staining. Arrow points to (glyco)protein of approximately 45 kDa, which is specifically purified with Ig-LC beads; * albumin (present in lanes 1 and 2); ** Ig-LC (present in lane 2).

FIG. 20: RT-PCR was used to detect FcRn mRNA in several cell types. PCR was performed with 1 μl and 3 μl of cDNA per 50 μl reaction volume. Human cell line K562 was compared with duplicates of mouse BMMCs and PMCs. A plasmid containing cloned human FcRn cDNA was used as a positive control. The PCR reactions all resulted in DNA fragments that were estimated to have the correct size of 347 bp, as was judged from comparison with the DNA marker bands on the left.

FIG. 21: Flow-cytometric analysis of K562 cells labeled with human kappa Ig-LC. Briefly, 10⁵ cells were washed once in FACS-buffer and subsequently incubated without any antibody, or in the presence of 1 or 3 μg of fluorescein- (FITC-) labeled kappa Ig-LC monomers. The cells were washed once with FACS buffer (PBS/5% FCS/0.1% sodium azide) and analyzed on a FACS Calibur flow cytometer. The same shift in fluorescent intensity was obtained when FITC-labeled kappa Ig-LC dimers were used instead of the monomers.

FIG. 22: Flow-cytometric analysis of human PMNs labeled with human kappa Ig-LC. Briefly, cells were incubated without any stimulus or in the presence of PMA (500 ng/ml, five minutes 37° C.) or fMLP (10⁻⁷ M, ten minutes 37° C.). For every sample, 10⁵ cells were washed once in FACS buffer and subsequently incubated without any antibody (no ab) or in the presence of 5 μg of fluorescein (FITC) labeled kappa Ig-LC (LC-FITC). The cells were washed once with FACS buffer (PBS/5% FCS/0.1% sodium azide) and analyzed on a FACS Calibur flow cytometer.

FIG. 23: The interaction between soluble hFcRn and biotinylated kappa (κ1, κ2), lambda (λ) and IgG was studied in an ELISA. Binding of different Igs was tested at pH 7.4 (n=2).

FIG. 24: Passive cutaneous anaphylaxis induced after local sensitization with Der p 2-specific light chains (filled symbols) followed by systemic challenge with (Der p 2-containing) house dust mite (HDM) extract. Ears were injected with PBS (open symbol) or Der p 2-specific light chains at 20 hours before intravenous injection with HDM extract. Ear thickness was measured 60 minutes after challenge.

FIG. 25a: Passive cutaneous anaphylaxis induced after local sensitization with Der p 2-specific light chains (filled symbols) followed by systemic challenge with recombinant Der p 2 protein. Ears were injected with PBS (open symbol) or Der p 2-specific light chains (closed symbols) at 20 hours before intravenous injection with recDer p 2. Ear thickness was measured 60 minutes after challenge.

FIG. 25b: Pretreatment with F991 (50 microgram i.p. at four hours before challenge) prevents development of ear swelling in Der p 2-induced passive anaphylaxis. Ears were injected with PBS (open symbol) or Der p 2-specific light chains at 20 hours before challenge by intravenous injection of recombinant Der p 2 protein. Four Hours before challenge, mice received 50 micrograms F991 intraperitoneally. Ear thickness was measured 60 minutes after challenge.

### DETAILED DESCRIPTION OF THE INVENTION

In one aspect, the invention provides evidence for a novel role for free Ig-LC. We show that Ig-LC transfer hapten sensitivity into naive animals and subsequent antigen challenge elicits an immediate hypersensitivity-like response, which appears to be mast cell dependent.

Mast cells have been implicated in a wide variety of biological responses including immediate hypersensitivity reactions, bacterial sepsis and also in T-cell-dependent reactions such as contact hypersensitivity reactions, experimental autoimmune encephalomyelitis (EAE), or non-allergic asthma. The diseases like contact sensitivity, EAE and non-allergic asthma can be induced by local delayed-type hypersensitivity (DTH) reactions and are independent of IgE or IgG1. Studies in mast cell-deficient animals show that mast cells are crucial in orchestrating a full DTH response. Humoral factors released by B-cells seem important in contact hypersensitivity reactions since hypersensitivity is impaired in B-cell-deficient animals. In ongoing studies, we have found that TNP-specific Ig-LC are capable of rescuing impaired contact hypersensitivity reactions as a result of hapten application in B-cell-deficient mice. In addition, passive sensitization with Ig-LC can give rise to a rapid and profound airway bronchoconstriction in mice after intra-airway antigen challenge, a reaction dependent on mast cell activation. Importantly, Ig-LC are involved in delayed-type hypersensitivity reactions leading to bronchoconstriction, cellular influx in bronchoalveolar lavage, and airway hyper reactivity after active sensitization with low-molecular weight compounds followed by intranasal challenge (manuscript in preparation). It is of interest that the secretion of Ig-LC is augmented under pathological conditions such as multiple sclerosis, Sjögren's disease, systemic lupus erythematosus, and other neurological disorders (18-21). For example, production of Ig-LC in patients with multiple sclerosis is greatly enhanced (20, 22). This production of Ig-LC is associated with recent antigenic stimulation and correlates with severity of the disease. In concord with our hypothesis, it has been demonstrated that mast cells play an important role in the pathogenesis of MS or EAE; substantially reduced disease symptoms are found in mast cell-deficient animals.

### EXAMPLES

### Example 1

### Ig-LC do not Activate Gamma Chain-Associated Receptors

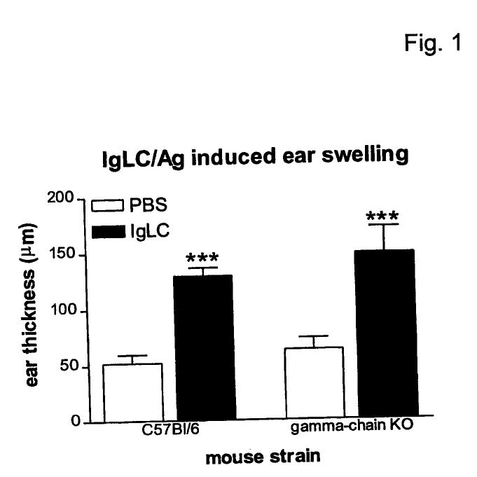

From our studies, it is clear that mast cells are crucial for the development of acute responses in skin and airways leading to ear swelling and acute bronchoconstriction, respectively. Thus far, triggering the high-affinity IgE receptor (FcεRI) and the low-affinity IgG-receptor (FcγRIII) are the only routes known to activate mast cells in an antigen-specific manner. We investigated whether Ig-LC exerted their action via activation of the FcγRIII or FcεRI receptors. Both receptors signal via the common gamma chain and can trigger hypersensitivity reactions via activation of mast cells. Passive sensitization of animals deficient in the common gamma chain (FcRγ−/−) resulted in similar ear swelling responses after hapten challenge as compared to wild-type (C57BL/6) animals (FIG. 1). This indicates that Ig-LC interacts with a receptor that does not need the common gamma chain for signaling and thereby excludes FcεRI and FcγRIII but also other gamma chain-associated receptors such as PIR-A and -B as effector molecules in Ig-LC-induced hypersensitivity reactions. Preliminary experiments showed that Ig-LC bind to mast cells and recognize antigen in rosetting assays with hapten-conjugated red blood cells. FACS analysis indicated that both IgE and IgG1 do not compete with the binding of Ig-LC to murine bone marrow-derived mast cells, confirming our results in the FcRγ−/− mice. Various attempts to directly activate murine bone marrow-derived mast cells in vitro to release prestored mediators after cross-linking of surface-bound Ig-LC were not successful. Although in some experiments a significant degree of degranulation was found, results were highly variable. The latter may be explained by a possible lack of co-stimulatory factor in vitro, a maturation-dependent expression of cell surface proteins involved in Ig-LC-mediated activation, or the simultaneous presence of inhibitory and activator receptors.

### Example 2

### Biochemical Isolation and Purification of Ig-LC Receptor

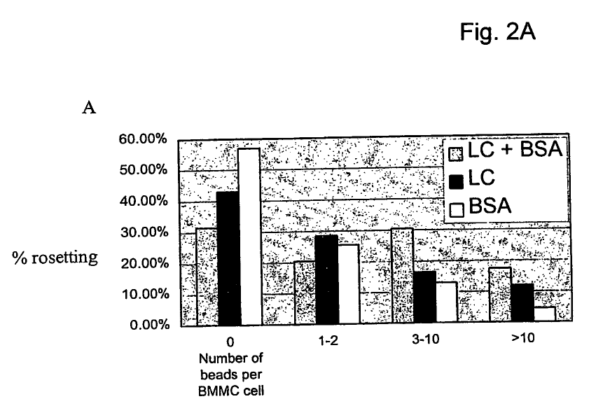

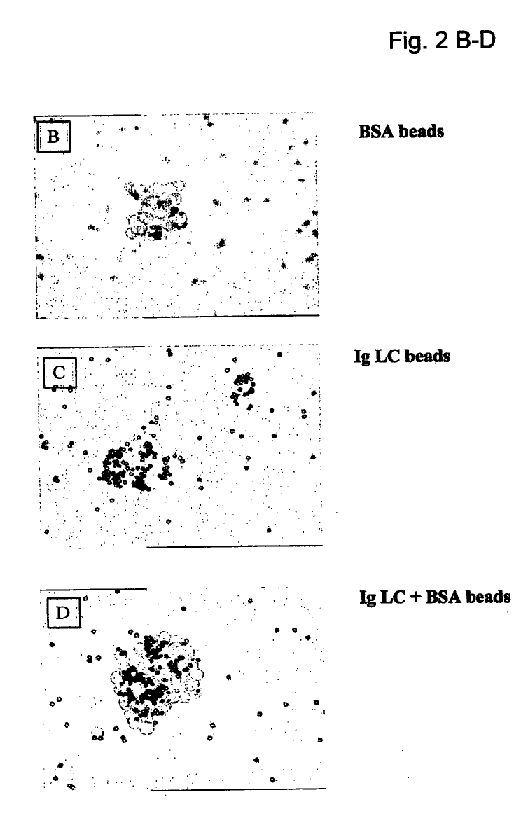

Chemical cross-linking of ligand to cellular membrane was the method employed to isolate and identify cell surface proteins as putative receptors for various ligands. Magnetic beads were coupled to Ig-LC. These beads were then incubated with murine bone marrow-derived mast cells and after washing the cells, Ig-LC (bait) were chemically cross-linked to cell surface proteins in immediate proximity to the binding site. After lysing the cells, Ig-LC cross-linked cell surface proteins were separated from non-bound proteins using a magnetic device. Proteins were washed and separated using SDS-PAGE (1-D or 2-D), followed by silver staining. Proteins were further characterized and identified with Maldi-TOF mass spectrometry and/or Edman degradation. Binding of Ig-LC-conjugated magnetic beads coupled to mast cells were visualized by light microscopy. Binding was specific for light chain, since no binding was detected when beads were conjugated to bovine serum albumin (BSA) (FIGS. 2A-2D and 19).

### Example 3

### Expression Cloning of the Ig-LC Receptor

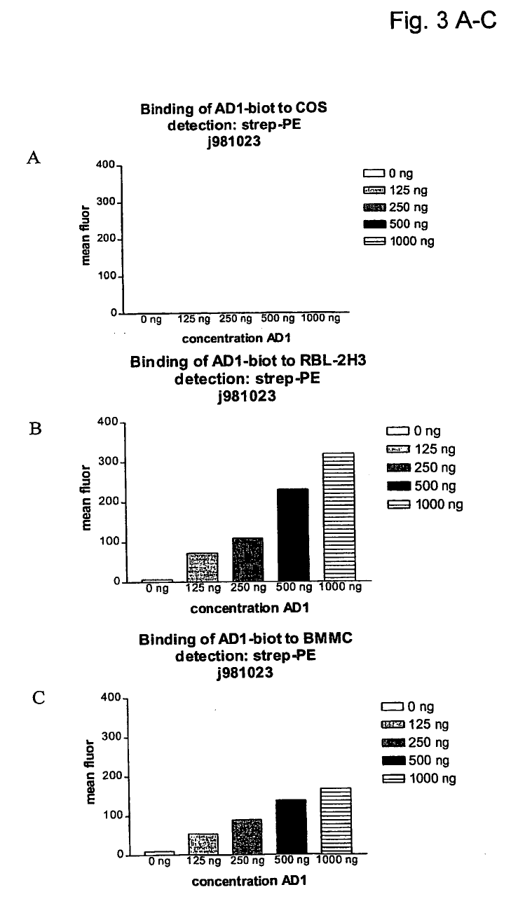

Expression cloning was used to clone an Ig-LC receptor. A cDNA library from primary cultured murine mast cells (BMMC) was constructed. This cDNA was transfected into mammalian cells. Transfected cells were compared in their binding capacity for Ig-LC using a flow cytometer. Cells binding Ig-LC above background were collected by the FACS sorter. Transfected DNA from these cells were isolated and used for succeeding transfection rounds. After several transfection/sorting rounds, a single Ig-LC receptor-expressing cell population was isolated. The transfected DNA from this population encodes for the putative Ig-LC receptor. A receptor present on mast cells of specific interest for binding Ig-LC is CD 63, a transmembrane-5 (TM5) membrane protein. This receptor is expressed by, for example, mast cells, granulocytes and leucocytes and cross-linking of this receptor results in mast cell activation and mediator release. FACS experiments showed the presence of CD63 on mast cell line RBL-2H3, but not on COS cells (FIG. 3).

CD63 seems widely expressed on different mammalian cells. In mast cell studies, CD63 has been used as an activation marker, since expression is increased after degranulation. On the other hand, a study in human CD63-transfected rat basophilic cells showed cross-linking of CD63 results in mast cell activation and degranulation. Thus far, no ligand for CD63 has been described yet (i.e., orphan receptor).

Ig Light Chain Receptor Binding Study:

In our current studies, we have shown that Ig light chain-mediated hypersensitivity responses can be effectively blocked by a 9-mer peptide (AHWSGHCCL). This peptide binds free light chains and thus prevents binding of the light chain to its receptor. It is, therefore, likely that the prominent binding sequence of this peptide, which is determined by the CCL part of the peptide, is present in the receptor for this ligand. Sequence analysis revealed that a sequence CCL or CCI is conserved in TM4 proteins such as CD63. Importantly, this sequence is present in an extracellular loop of the protein and, therefore, a putative docking site for receptor ligands. CD63 is the most likely candidate of this TM4 family because of its known expression in mast cells and its demonstrated capability to activate mast cells.

### Example 4

### Role of Mast Cells in Ig-LC Responses

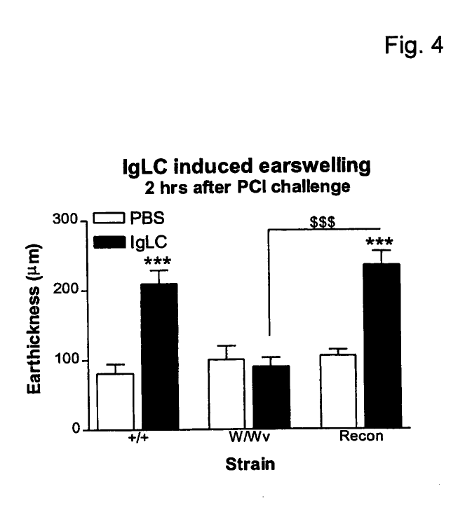

The involvement of mast cells in eliciting ear swelling responses after topical challenge of Ig-LC-sensitized mice was investigated using mast cell-deficient mice (W/Wv). As shown in FIG. 4, passive sensitization of mast cell-deficient mice with TNP-specific Ig-LC followed by topical application of TNP did not result in a significant ear swelling at two hours after challenge. As expected, similarly treated control mast cell-sufficient littermates (+/+) showed normal ear swelling responses. To further prove that the absence of mast cells alone was responsible for the completely reduced ear swelling response in the mast cell-deficient mice, we reconstituted these animals with bone marrow-derived mast cells from wild-type animals. Reestablishment of the mast cell population by local injection of bone marrow-derived mast cells restored the ear swelling responses in Ig-LC-sensitized and hapten-challenged animals (FIG. 4).

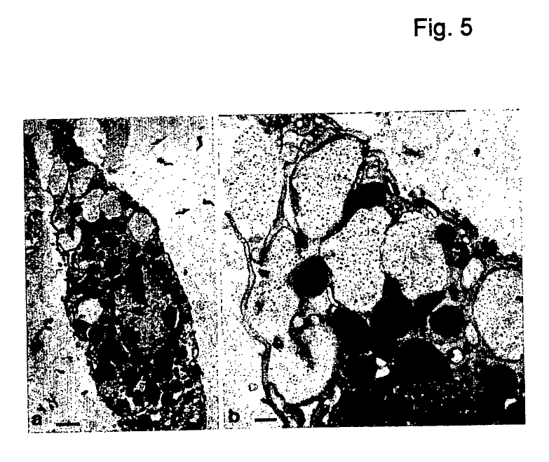

Indeed, hapten challenge of Ig-LC-sensitized animals was accompanied with a rapid increase in plasma histamine levels in vivo and direct proof for mast cell activation was obtained after histological and ultrastructural analysis of biopsies of the ears at one hour after topical hapten challenge. Mast cells in tissue sections of mice intravenously sensitized with TNP-specific Ig-LC showed marked signs of degranulation after re-exposure to hapten. Electron microscopy revealed that degranulation was characterized by swelling of intracytoplasmic granules, decrease of electron-density of the granules and extrusion of membrane-free granules from the mast cells (FIG. 5).

### Example 5

### Specific Recognition of Antigen by Ig-LC

In general, it is acknowledged that both heavy chains and light chains contribute to binding of antigens by immunoglobulins, which are monomers or multimers of a tetrameric structure consisting of two light chains and two heavy chains. The structure of an Ig-LC can be separated in a constant and variable region. The latter region contains hypervariable domains (CDRs) that are responsible for antigen recognition. The genes encoding these regions undergo rearrangement/mutation to effect affinity maturation and gain specificity for a certain antigen. Similar mechanisms play a role in shaping the antigen recognition by Ig heavy chains.

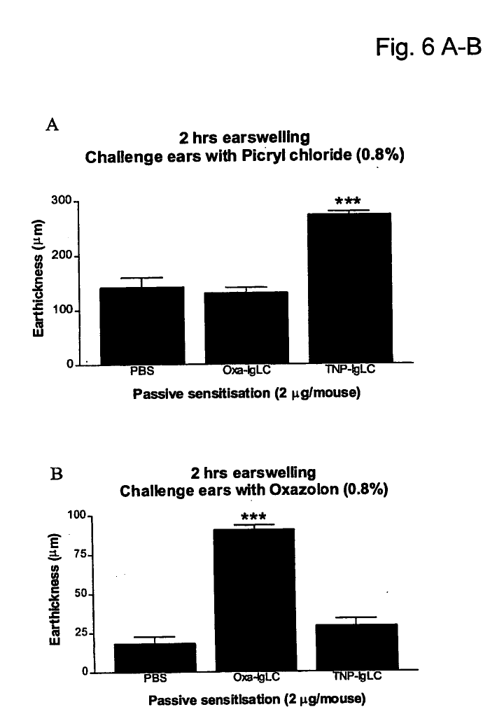

In order to prove that free Ig-LC have the capability to recognize and bind to antigen, we have performed the following experiments. First, two Ig-LCs with different antigen specificity were generated by separation of immunoglobulin heavy and light chains from oxazolone- and TNP-specific IgGs. Naive mice were sensitized with the isolated Ig-LC and subsequently topically challenged with TNP or oxazolone. Two hours after challenge, ear thickness was measured. As shown in FIG. 6, when mice were sensitized with oxazolone-specific Ig-LC, only an increase in ear thickness was measured when the ears were challenged with oxazolone and not after application of picryl chloride, i.e., TNP. Vice versa, when animals were sensitized with TNP-specific Ig-LC, ear swelling was only induced by TNP and not by oxazolone.

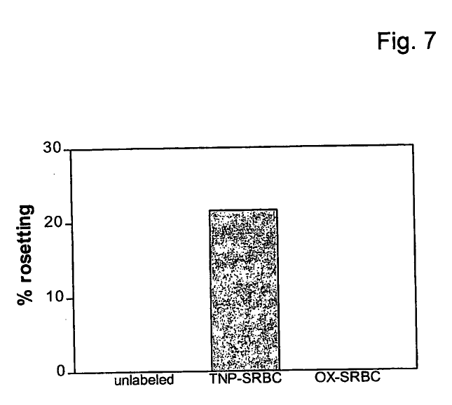

In a second experiment, we investigated if Ig-LC, when bound to mast cells, is able to recognize antigen in a proper way. In in vitro experiments, bone marrow-derived mast cells were sensitized with TNP-specific Ig-LC. Next, sensitized cells were co-incubated with either unlabeled, TNP-labeled or oxazolone-labeled sheep red blood cells (SRBC). Binding (resetting) of the SRBC was scored under a light microscope. As evidenced in FIG. 7, TNP-specific Ig-LC-sensitized mast cells only show significant binding to TNP-labeled SRBC, but not to unlabeled or with an unrelated hapten (OX)-coupled SRBC. This experiment confirms that Ig-LC are indeed able to specifically recognize antigen.

### Example 6

### Production of Ig-LC After Contact Sensitization of Mice

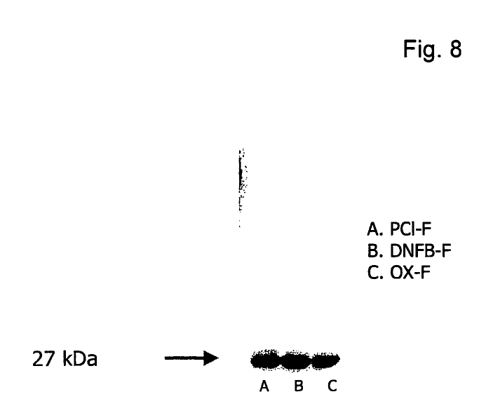

Induction of immediate hypersensitivity by Ig-LC may be physiologically relevant. Ig-LC are produced and secreted upon antigen exposure. To test this hypothesis, spleen and lymph node cells from mice that had been sensitized epicutaneously to PCl, DNFB or oxazolone four days before, were cultured in vitro for one day in the absence of hapten. Hapten-binding proteins from culture supernatant were isolated by hapten-affinity chromatography. Western analysis showed that the hapten-binding factors obtained this way contained Ig kappa light chain (FIG. 8), but no Ig heavy chains were detected (data not shown). Importantly, TNP-binding factor could be isolated from culture supernatant of spleen and lymph node cells isolated from mice as early as one day after topical sensitization with PCl. In this preparation, the presence of Ig-LC was confirmed by Western blot analysis (data not shown). The N-terminal amino acid sequence of this protein showed almost complete homology with mouse Ig kappa light chain.

### Example 7

### Measurement Ig-LC in Human Serum

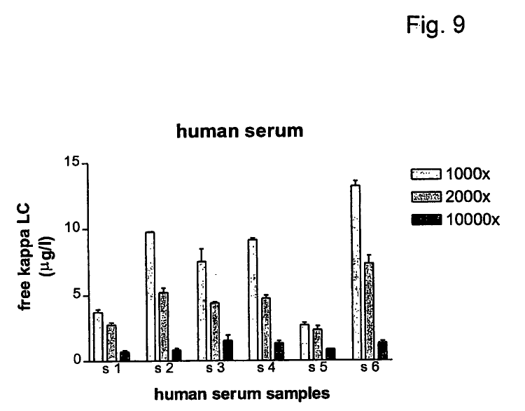

Free Ig-LC can be detected in various human body fluids, e.g., liquor, urine and serum. Using an Ig-LC-specific ELISA, we were able to detect significant levels of Ig-LC in serum (FIG. 9). The detected levels are comparable to those reported earlier by other groups.

### Example 8

### Effects of F991, an Ig-LC-Binding Peptide

F991 is developed as an antagonist of Ig light chain. The working hypothesis for this compound is that it binds to Ig light chains and thereby prevents binding of light chains to their putative receptors. F991 is a 9-mer peptide derived from the endogenous protein uromodulin. The potency of F991 to inhibit Ig-LC-induced cutaneous reactions (ear swelling responses) was studied. Our working hypothesis for this compound is that it is an antagonist of Ig-LC and binds to Ig-LC and thereby prevents binding of Ig-LC to their putative receptors. F991 was administered in different dosages, via various administration routes.

A. Dose-Dependent Inhibition After Intravenous Administration.

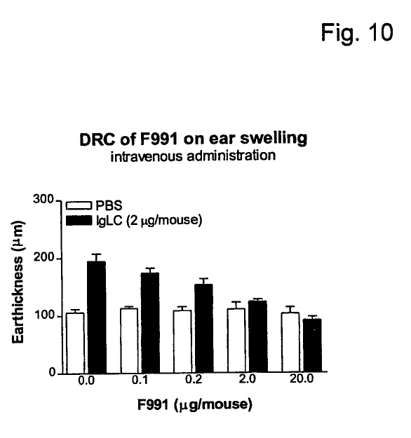

Indicated amounts of F991 per mouse were intravenously injected at 30 minutes before hapten challenge. At that same time point, mice were injected (passively sensitized) with TNP-specific Ig-LC or vehicle (PBS). Two hours after hapten challenge, increase in ear thickness was monitored. FIG. 10 shows a clear dose-dependent inhibition of the ear swelling response by F991. Amounts of 2 μg per mouse (1.9 nmole!!) were sufficient to completely block the Ig-LC-induced ear swelling (FIG. 10).

B. Other Administration Routes: Intraperitoneal Administration of F991

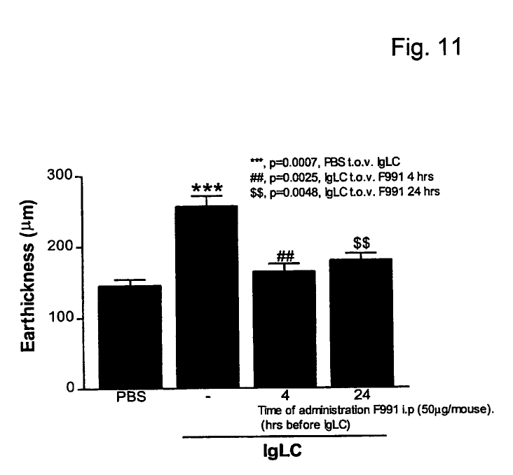

Mice were injected with 50 μg of F991 intraperitoneally at four or 24 hours before challenge with hapten. Thirty minutes before challenge, the animals were passively (i.v.) sensitized with TNP-specific Ig-LC and two hours after hapten application onto the ears, ear thickness was measured. As demonstrated in FIG. 11, i.p. administration of F991, even at 24 hours before hapten challenge, completely prevented the induction of an ear swelling response (FIG. 11).

C. Subcutaneous Administration of F991

Protocol (see above) for i.p. administration, except F991, was injected subcutaneously.

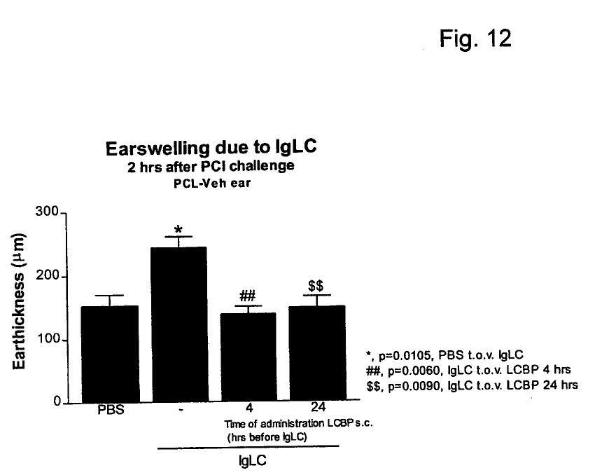

Similar to the i.p. administration of F991 when injected subcutaneously, F991 again completely inhibited Ig-LC-induced ear swelling after hapten challenge (FIG. 12).

### Example 9

### Epicutaneous Application of F991 (in Ointment)

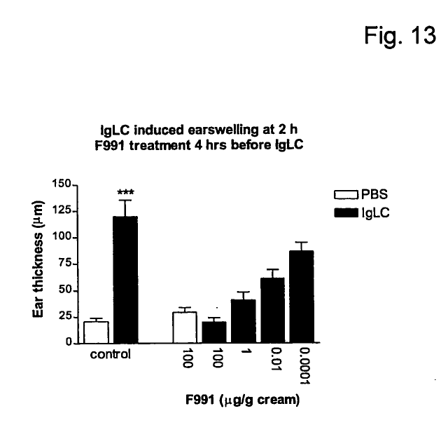

A. F991 was prepared as an ointment in Cremor Cetomacrogolis FNA and topically applied on the ear at four hours before hapten challenge. Subsequently, mice were passively sensitized with 2 μg TNP-specific Ig-LC at 30 minutes before and ear thickness was measured two hours after hapten challenge of the ears. As shown in FIG. 13, topical application of F991 in an ointment resulted in a dose-dependent inhibition of the ear swelling induced after hapten challenge of Ig-LC-sensitized animals (FIG. 13).

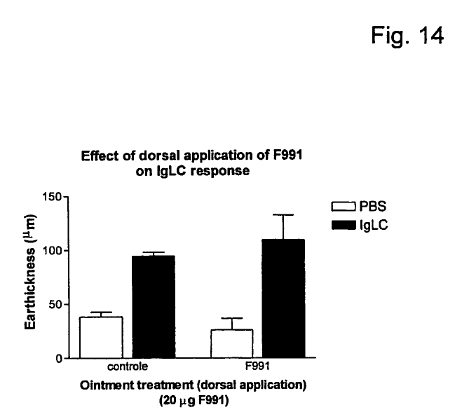

B. We further investigated if local application of F991 as an ointment resulted in systemic inhibition of the Ig-LC-induced effects. Therefore, F991 was applied on the back of the mice instead of at the site of hapten challenge (ear). Mice were again sensitized and challenged as described above. FIG. 14 shows that topical application of F991 does not result in systemic inhibition of the Ig-LC effects. This means that epicutaneous treatment should be done at the site of challenge (FIG. 14).

C. Stability of F991 in Ointment

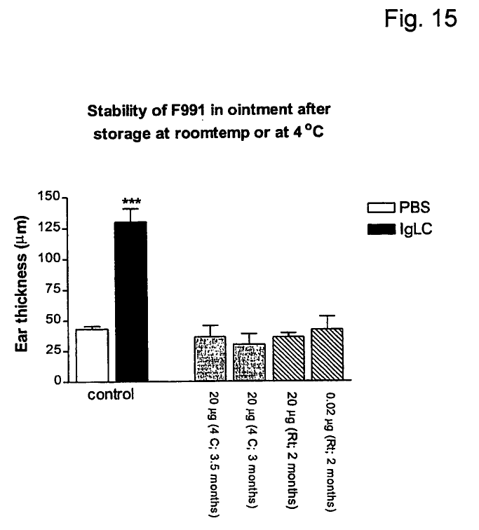

To determine the stability of F991 in ointment, different preparations of F991 in Cremor Cetamacrogolis FNA were tested for their activity after storage for two to three months at room temperature and at 4° C. The different preparations were applied at the ear as described in A (above) and subsequently, ear swelling was monitored after hapten challenge of Ig-LC-sensitized animals. As shown in FIG. 15, F991 stored under all different conditions retained its activity (FIG. 15).

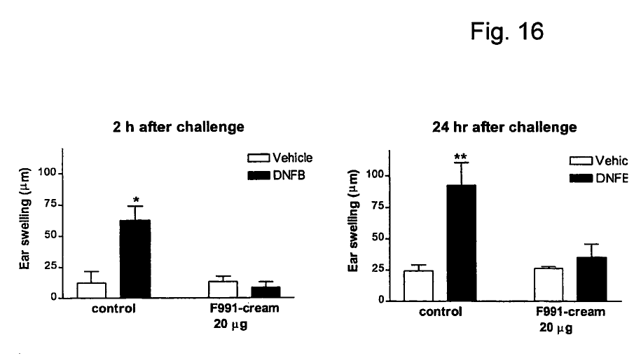

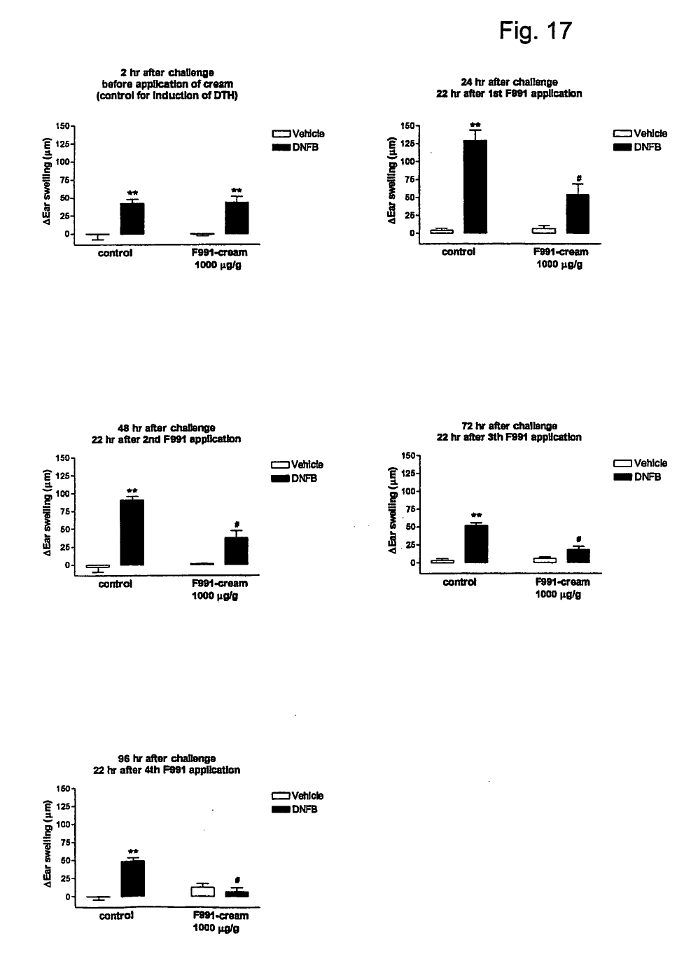

D. Next, we investigated if topical treatment with F991 also inhibited the development of contact sensitivity reactions induced after active sensitization. Mice were sensitized with a low-molecular weight compound DNFB on the skin and footpads (on day 0 and 1). Five days after the start of sensitization and four hours before local challenge with hapten, mice were treated with F991 on the ears. Two and 24 hours after hapten challenge, the increase in ear thickness was determined. As shown in FIG. 16, topical treatment with F991 completely inhibited the ear swelling at both two and 24 hours after challenge. This experiment indicates that topical treatment with F991 may be of therapeutic use in the treatment of contact dermatitis, a disease with similar characteristics (FIG. 16).

E. Next, we investigated if topical treatment with F991 also inhibited or decreased the development of contact sensitivity reactions induced after active sensitization. Mice were sensitized with a low-molecular weight compound DNFB on the skin and footpads (on day 0 and 1). Five days after the start of sensitization and two hours after local challenge with hapten, mice were treated with F991 on the ears. Ear swelling was measured 22 hours after application of the cream. Subsequently, mice were treated daily with the F991 cream, two hours after measuring of the ear swelling. Control mice were treated with the vehicle cream without F991. Two, 24, 48, 72 and 96 hours after hapten challenge, the increase in ear thickness was determined. As shown in FIG. 17, topical treatment with F991 diminished the ear swelling at all time-points after two hours after challenge. This experiment indicates that topical treatment with F991 may be of therapeutic use in the treatment of contact dermatitis, a disease with similar characteristics.

### Example 10

### Ig-LC, Mast Cells and Multiple Sclerosis

It is well documented that production of Ig-LC in patients with multiple sclerosis is greatly enhanced. Elevated levels of free Ig-LC can be detected in cerebrospinal fluid and urine of MS patients. This production of free Ig-LC is associated with recent antigenic stimulation and correlates with the severity of the disease. In concord with our hypothesis, it has been demonstrated that mast cells play an important role in the pathogenesis of MS; substantially reduced disease symptoms are found in mast cell-deficient animals (23). Further, mast cells are observed in CNS plaques, and histamine and tryptase levels are elevated in the liquor of MS patients, whereas treatment with mast cell stabilizers or antagonists of histamine and serotonin seem to ameliorate MS.

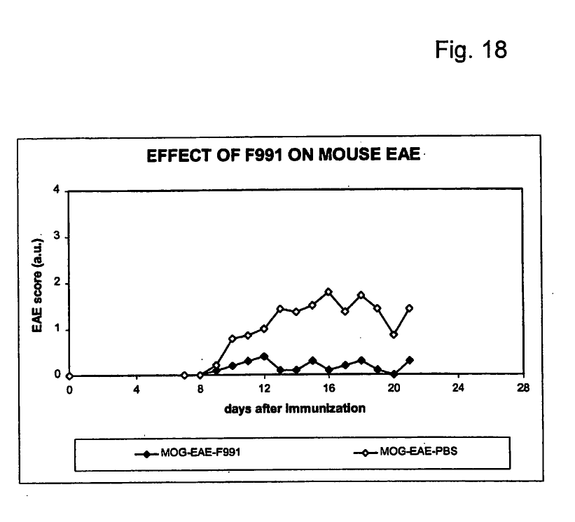

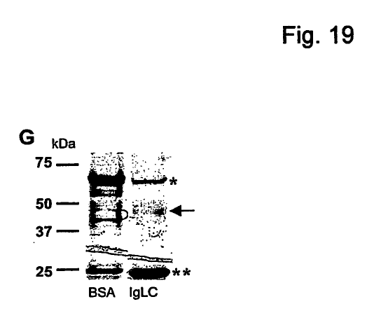

If light chains were involved in the activation of mast cells in patients with MS, the prediction is that F991 may be of therapeutic interest in the treatment of MS. We tested to see if F991 was able to prevent or reduce development of clinical signs in an established mouse model for MS, i.e., myelin oligodendrocyte glycoprotein (MOG)-induced experimental allergic encephalomyelitis (EAE). Mice were sensitized with antigenic peptide 35-55 from MOG in complete freund adjuvant. At the day of sensitization and three days later, mice also received an injection with pertussis toxin. In general, ten to twelve days after the start of sensitization, mice developed clinical signs of MS, which can be scored in degree of paralysis (0=no paralysis, 1=tail flaccidity, 2=hind limb weakness, 3=hind limb paralysis, 4=forelimb paralysis or loss of ability to right supine, 5=death). To investigate if F991 is able to prevent development of clinical signs of MS, mice were daily injected with 50 μg F991/animal i.p. starting at day −1 until day 21. As shown in FIG. 18, treatment with F991 completely prevented the development of clinical signs of paralysis. Control mice developed disease with a mean day of onset of 11.8 and a mean clinical score of 2.3. There was a significant difference in disease burden (mean cumulative EAE score) between the F991-treated group (mean 4.2) and the control group (mean 18.3). Termination of the treatment with F991 at 21 days after sensitization, resulted in the development of some minor clinical symptoms (“silly walk”), but not in a clear manifestation of EAE/MS (data not shown) (FIG. 18).

### Example 11

### FcRn Receptor Binding to Ig-LC

1. Expression of FcRn in Mouse Mast Cells

A RT-PCR reaction was performed to detect FcRn mRNA in mouse bone marrow-derived mast cells (BMMC) and pulmonary mast cells (PMC).

Method

TRIzol reagent (Gibco) was used to isolate total RNA from four-week-old BMMCs and PMCs and cultured K562 erythroleukemia cells. The RNA isolations and subsequent amplification steps from BMMCs and PMCs were performed as duplicates. First strand cDNA was synthesized from 1.6 μg total RNA by using SuperScript reverse transcriptase (Invitrogen) and an oligo dT primer. PCR reactions were done with 1 μl and 3 μl of first strand cDNA per 50 μl reaction volume. The forward and reverse primers for the PCR reactions had the following DNA sequences, respectively: CCTGCTGGGCTGTGAACTGG (SEQ ID NO:2) and GCTCCGGDGGGTAGAAGGAG (SEQ ID NO:3). Using these primers, a DNA fragment of 347 basepairs (bp) was expected to be amplified from both mouse and human sequences. DNA sequences were run on 1.5% agarose gels and visualized by ethidium bromide staining.

Results

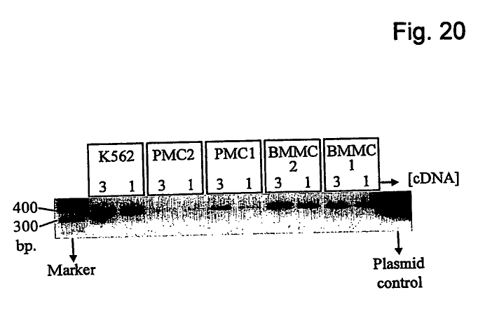

The main amplified DNA fragment that was obtained after doing the RT-PCR reaction on the RNA isolated from BMMCs, PMCs, K562 cells, and a control plasmid that contained cloned human FcRn cDNA as an insert, had the expected size of approximately 347 bp (FIG. 20). The results indicate that mRNA encoding FcRn is present in PMCs as well as in the BMMCs, although the concentration in BMMCs is a little higher. In K562 cells, even more amplification product was obtained, suggesting that these cells express relatively high levels of FcRn.

Conclusions

It should be noted that these experiments are not conclusive about the mRNA expression levels, since no household mRNA level has been determined to ensure that equal amounts of cDNA have been added to each PCR reaction. However, the experiments confirm the presence of FcRn mRNA in mouse BMMCs and PMCs.

2. Presence of FcRn on the Outside of Mast Cells

From literature, it is known that expression of FcRn in cells does not guarantee the presence of the protein on the outside of the cell. The co-expression of β-2-microglobulin is required to transport FcRn to the plasma membrane. The lack of an antibody against mouse FcRn kept us from confirming the presence of FcRn on the outside of the BMMCs.

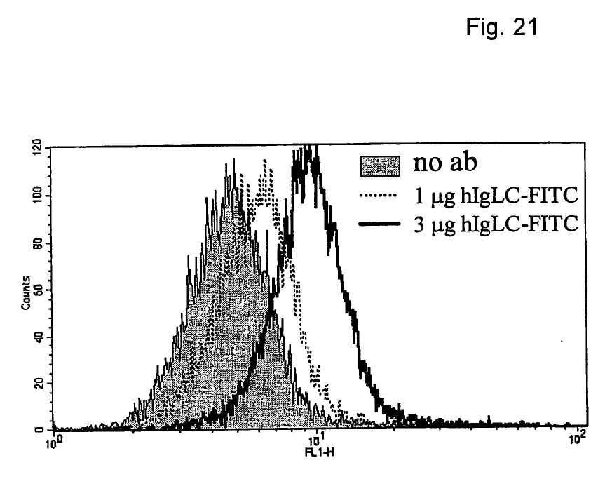

Immunofluorescence microscopy has been used to show that FcRn is present intracellularly, as well as on the outside of the plasma membrane of K562 cells. Flow-cytometric analysis shows that K562 cells are able to bind human kappa Ig-LC (FIG. 21). The presence of FcRn on the outside of the cell in combination with the ability to bind Ig-LC is still in agreement with the proposed role of FcRn as the LC-R, although it does not prove this.

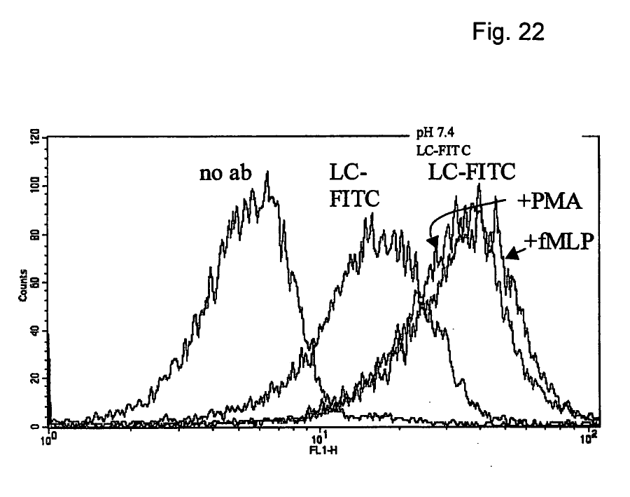

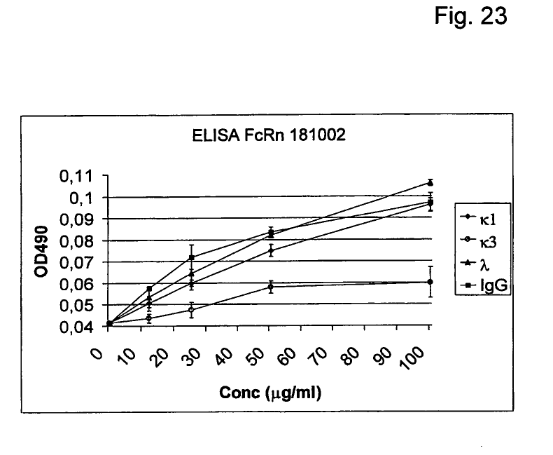

Flow cytometry was also used to show binding of Ig-LC to freshly isolated human neutrophils (PMNs) (FIG. 22). This binding was enhanced after stimulation of PMNs by PMA or fMLP. It is known from literature that most FcRn is present in intracellular vesicles, which fuse with the plasma membrane after stimulation. This finding again matches with the proposed role of FcRn as the LC-R, but does not prove it.

3. Interaction Between FcRn and Ig-LC

An ELISA was used to study a possible interaction between human FcRn (hFcRn) and human κ-Ig-LC.

Method

Two different types of human κ-Ig-LCs (κ1 and κ3), a λ-Ig-LC, and a mixture of IgGs were chemically linked to biotin. The Igs were dissolved in PBS at a concentration of 1 mg/ml. The Ig-LCs were incubated in the presence of 426 μg/ml EZ-link Sulfo-NHS-LC-Biotin (Pierce) at room temperature in the dark for 90 minutes. The reaction of the IgGs was performed with 220 μg/ml biotinylation agent for 30 minutes. The labeled Igs were dialyzed against PBS for 12 hours. An ELISA plate was coated with 100 ng/ml recombinant soluble hFcRn (a kind gift of P. Bjorkman) in PBS for 15 hours at 4° C. Blocking of the plate was done with 1% BSA in PBS/0.5% Tween-20 for 60 minutes at room temperature, followed by three washes with washing buffer (PBS containing 0.05% Tween-20). The plate was incubated with various amounts of biotinylated Igs in assay buffer (washing buffer with 0.1% BSA) for 60 minutes at room temperature. After three washes, the plate was incubated with Streptavidin Poly-HRP (100 ng/ml in assay buffer) for 30 minutes, followed by six washes. The plate was assayed with o-phenylenediamine according to the protocol of the manufacturer.

Results

Several ELISA experiments showed a significant binding of Ig-LCs and IgG to the hFcRn. An example is shown in FIG. 22. No binding was seen when the hFcRn was omitted from the coating reaction, suggesting that the interaction is specific (not shown). The amount of signal differs between the Igs, but this is not conclusive since the biotinylation efficiency of each sample has not been determined. However, there are some differences in the shape of the binding curves, with IgG and κ3 reaching a plateau at lower protein concentrations than κ1 and λ. It should be noted that, despite the observed specificity, there were no differences in binding between incubations at pH 7.4 or 6.5 (not shown). This contradicts findings in literature, where several groups independently showed binding of IgG at pH 6.5, but not at pH 7.4.

Conclusion

In the described ELISA setup, a significant binding of Ig-LCs to hFcRn was shown. However, further research using another experimental method will be required to confirm and validate this observation.

### Example 12

### Clinical Safety Study of a Phase 1 Single, Rising Dose, Double-Blind, Placebo-Controlled Study with F991 in Healthy Male Volunteers

Summary:

Objectives

Primary objective: to study the safety and tolerability of F991 at increasing single dose levels in healthy male volunteers

Secondary objective: to study the pharmacokinetics of F991 in healthy male volunteers

Tertiary objective: to study the pharmacodynamics of F991 in healthy male volunteers (exploratory)

Methodology

Design: This study was a double-blind, placebo-controlled, single rising dose study in two alternating panels of eight healthy male volunteers. In each panel, two volunteers received placebo throughout the study and the other six volunteers received three increasing single doses of F991.

Procedures and Assessments

Screening and follow-up: clinical laboratory, full physical examination, ECG; at eligibility screening: medical history, drug screen including alcohol, HBsAg, anti-HCV and anti-HIV 1/2

Observation period: each period in clinic from −17 hours prior to drug administration up to 24 hours after drug administration

Blood sampling:
for pharmacokinetics: for total plasma F991:
   panel I period 1: pre-dose and at 5, 10, 15, 20, 30 and 45 minutes and 1, 1.5, 2, 3, 4, 5, 6, 8, 10, 12, 16 and 24 hours post-dose
   panel I period 2 and panel II periods 1 and 2: pre-dose and at 15, 30, 35, 40 and 50 minutes and 1, 1.5, 2, 3, 4, 5, 6, 8, 10, 12, 16 and 24 hours post-dose
   panel I period 3: pre-dose and at 15, 30, 45, 50, 55 and 65 minutes and 1.5, 2, 3, 4, 5, 6, 8, 10, 12, 16 and 24 hours post-dose
   panel II period 3: pre-dose and at 15, 30, 55, 60 and 65 minutes and 1.5, 2, 3, 4, 5, 6, 8, 10, 12, 16 and 24 hours post-dose
for pharmacodynamics: tryptase and Ig-LC (Immunoglobulin Light Chains) pre-dose and one hour and eight hours post-dose

Safety assessments: vital signs; adverse events (including eyesight); ECG four times each period; clinical laboratory: on admission for each period

Bioanalysis: by Sponsor

Volunteers

16 healthy male volunteers

Diagnosis and Main Criteria for Inclusion

Age: 18-55 yr, inclusive

Weight: within ±15% deviation from normal range, from 60 to 100 kg, inclusive

Gender: male

Study Medication

Active substance: F991

Activity: immunomodulating peptide

Indication: RA, asthma, inflammatory diseases

Strength: freeze-dried material (8.4 mg) to be reconstituted with 10 mL water/0.9% NaCl: 0.84 mg F991-peptide/mL, 10 mM citrate pH 5, 2.25% mannitol, 0.45% NaCl

Dosage form: iv infusion

Placebo: visually matching active substance

The following treatments were administered, as a single dose in alternating panels, according to the randomization schedule.

Panel I: period 1: 0.08 mg/kg F991 or placebo iv infusion
period 2: 0.8 mg/kg F991 or placebo iv infusion
period 3: 3.2 mg/kg F991 or placebo iv infusion

Panel II: period 1: 0.32 mg/kg F991 or placebo iv infusion
period 2: 1.6 mg/kg F991 or placebo iv infusion
period 3: 6.4 mg/kg F991 or placebo iv infusion
   Criteria for Evaluation

Safety: adverse events, clinical laboratory test results, ECG recordings, vital signs and physical examination

Pharmacokinetics: pharmacokinetic parameters derived from F991 plasma concentration-time data are: Cₘₐₓ, tₘₐₓ, kₑₗ, t_{1/2}, AUCₗₐₛₜ, AUC_{0-inf}, CL and V_{d}

Pharmacodynamics: pharmacodynamic parameters of tryptase and Ig-LC concentrations

Statistical Methods

Safety parameters: descriptive statistics

Pharmacokinetics parameters: descriptive statistics

Pharmacodynamic parameters: descriptive statistics

Results and Conclusions

No correlation was observed between the dose of F991 and the number, frequency and intensity of AEs. There were no SAEs during the study. All possibly related AEs were of mild intensity, except one, which was a headache of moderate intensity that lasted about 14.5 hours and required treatment with a single dose of 500 mg paracetamol. The most frequently reported AE with a possible relationship to the study drug was headache, however, this occurred mainly in the placebo group. For volunteers on active drug, the most frequently reported AEs were eye disorders. With regard to clinical laboratory data, vital signs, ECG and physical examination, no clinically significant abnormalities were observed. I.V. administration of single rising doses up to 6.4 mg/kg of F991 was safe and well tolerated in healthy male volunteers.

### Example 13

### The Effect of F91 1 on Allergen Reaction to House Dust Mite in Mice

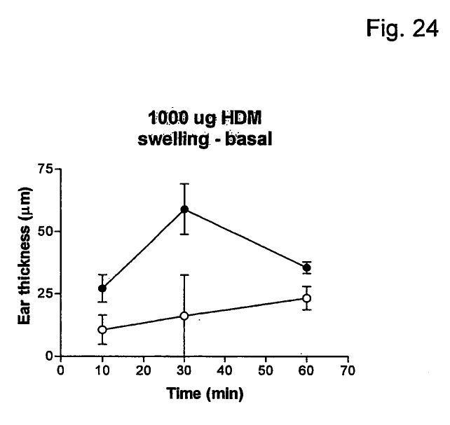

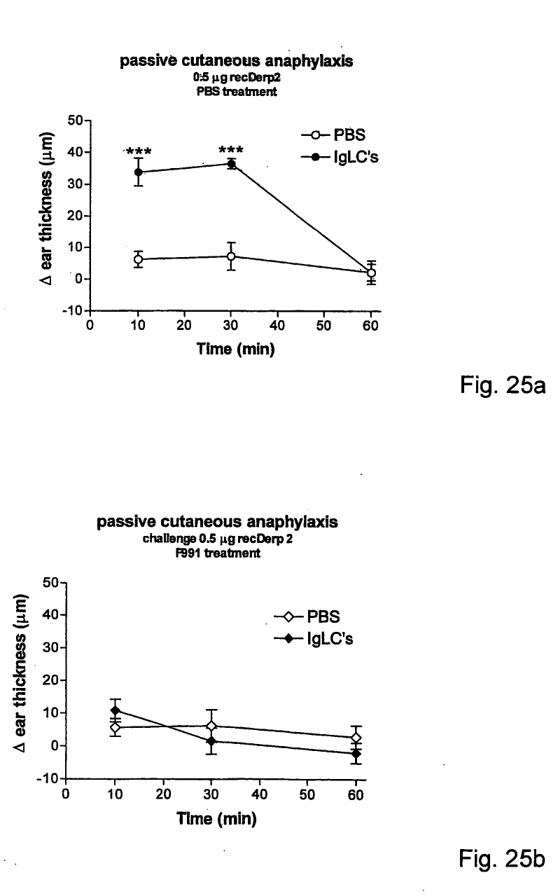

Next, we investigated if topical treatment with F991 also inhibited the development of contact sensitivity reactions induced after passive sensitization with house dust mite allergen Der p 2-specific IG-LC. Mice were sensitized with a Der p 2-specific IG-LC in the ear skin. Sixteen hours after sensitization and four hours before systemic challenge with house dust mite extract or recombinant Der p 2, mice were treated with F991 on the ears. Sixty minutes after allergen challenge, the thickness of the ear was determined. As shown in FIGS. 24 and 25, topical treatment with F991 completely inhibited the ear swelling 60 minutes after challenge. This experiment shows that topical treatment with F991 may be of therapeutic use in the treatment of, for example, cutaneous anaphylaxia and contact dermatitis.

### REFERENCES

1. Van Loveren, H., R. E. Ratzlaff, K. Kato, R. Meade, T. A. Ferguson, G. M. Iverson, C.

A. Janeway, and P. W. Askenase. Immune serum from mice contact-sensitized with picryl chloride contains an antigen-specific T-cell factor that transfers immediate cutaneous reactivity. Eur. J. Immunol. 16(10):1203-8 (1986).

2. Hopper, J. E., and E. Papagiannes. Evidence by radioimmunoassay that mitogen-activated human blood mononuclear cells secrete significant amounts of light chain Ig unassociated with heavy chain. Cell Immunol 101(1): 122-31 (1986).

3. Shapiro, A. L., M. D. Scharff, J. V. Maizel, and J. W. Uhr. Synthesis of excess light chains of gamma globulin by rabbit lymph node cells. Nature 211(46):243-5. (1966).

4. Skvortsov, V. T., and A. E. Gurvich. Relative rates of synthesis of immunoglobulins and light chains in rabbit spleen cells during secondary response. Nature 218(139):377-8 (1968).

5. Hannam-Harris, A. C., J. Gordon, and J. L. Smith. Immunoglobulin synthesis by neoplastic B lymphocytes: free light chain synthesis as a marker of B-cell differentiation. J. Immunol. 125(5):2177-81 (1980).

6. Hannam-Harris, A. C., and J. L. Smith. Free immunoglobulin light chain synthesis by human fetal liver and cord blood lymphocytes. Immunology 43(3):417-23 (1981).

7. Waldmann, T. A., W. Strober, and R. P. Mogielnicki. The renal handling of low-molecular weight proteins. II. Disorders of serum protein catabolism in patients with tubular proteinuria, the nephrotic syndrome, or uremia. J. Clin. Invest. 51(8):2162-74 (1972).

8. Yoo, T. J., O. A. Roholt, and D. Pressman. Specific binding activity of isolated light chains of antibodies. Science 157(789):707-9 (1967).

9. Masat, L., M. Wabl, and J. P. Johnson. A simpler sort of antibody. Proc. Natl. Acad. Sci. U.S.A. 91(3):893-6 (1994).

10. Sun, M., L. Li, Q. S. Gao, and S. Paul. Antigen recognition by an antibody light chain. J. Biol. Chem. 269(1):734-8 (1994).

11. Mahana, W., F. Jacquemart, and M. Ermonval. A murine monoclonal multireactive immunoglobulin kappa light chain. Scand. J. Immunol. 39(1):107-10 (1994).

12. Ledbetter, J. A., H. P. Fell, L. S. Grosmaire, N. A. Norris, and T. T. Tsu. An immunoglobulin light chain dimer with CD4 antigen specificity. Mol. Immunol. 24(12):1255-61 (1987).

13. Schechter, I., and E. Ziv. Binding of 2,4-dinitrophenyl derivatives by the light chain dimer obtained from immunoglobulin A produced by MOPC-315 mouse myeloma. Biochemistry 15(13):2785-90 (1976).

14. Painter, R. G., H. J. Sage, and C. Tanford. Contributions of heavy and light chains of rabbit immunoglobulin G to antibody activity. I. Binding studies on isolated heavy and light chains. Biochemistry 11(8):1327-37 (1972).

15. Tribbick, G., A. B. Edmundson, T. J. Mason, and H. M. Geysen. Similar binding properties of peptide ligands for a human immunoglobulin and its light chain dimer. Mol. Immunol. 26(7):625-35 (1989).

16. Thiagarajan, P., R. Dannenbring, K. Matsuura, A. Tramontano, G. Gololobov, and S. Paul. Monoclonal antibody light chain with prothrombinase activity. Biochemistry 39(21):6459-65 (2000).

17. Sun, M., Q. S. Gao, L. Li, and S. Paul. Proteolytic activity of an antibody light chain. J. Immunol. 153(11):5121-6 (1994).

18. Fagnart, O. C., C. J. Sindic, and C. Laterre. Free kappa and lambda light chain levels in the cerebrospinal fluid of patients with multiple sclerosis and other neurological diseases. J. Neuroimmunol. 19(1-2):119-32 (1988).

19. Moutsopoulos, H. M., A. D. Steinberg, A. S. Fauci, H. C. Lane, and N. M. Papadopoulos. High incidence of free monoclonal lambda light chains in the sera of patients with Sjögren's syndrome. J. Immunol. 130(6):2663-5 (1983).

20. Solling, K., J. Solling, and F. K. Romer. Free light chains of immunoglobulins in serum from patients with rheumatoid arthritis, sarcoidosis, chronic infections and pulmonary cancer. Acta. Med. Scand. 209(6):473-7 (1981).

21. Lamers, K. J., J. G. de Jong, P. J. Jongen, M. J. Kock-Jansen, M. A. Teunesen, and E. M. Prudon-Rosmulder. Cerebrospinal fluid free kappa light chains versus IgG findings in neurological disorders: qualitative and quantitative measurements. J. Neuroimmunol. 62(1):19-25 (1995).

22. Mehta, P. D., S. D. Cook, R. A. Troiano, and P. K. Coyle. Increased free light chains in the urine from patients with multiple sclerosis. Neurology 41(4):540-4 (1991).

23. Secor, V. H., W. E. Secor, C. A. Gutekunst, and M. A. Brown. Mast cells are essential for early onset and severe disease in a murine model of multiple sclerosis. J. Exp. Med. 191(5):813-22 (2000).

1. An isolated cell comprising an immunoglobulin free light chain (Ig-LC) receptor capable of activating a signal transduction pathway in said isolated cell upon binding of an Ig-LC to a receptor, wherein said signal transduction pathway is independent of the presence of a functional common gamma chain-associated receptor on said isolated cell.

2. The isolated cell of claim 1, wherein said isolated cell comprises a gamma chain receptor-deficient cell.

3. The isolated cell of claim 1, wherein said signal transduction pathway in said isolated cell is activated upon binding of an Ig-LC to said receptor and subsequent cross-linking of receptor-bound Ig-LC.

4. The isolated cell of claim 1, wherein said isolated cell comprises a mast cell.

5. The isolated cell of claim 1, wherein said receptor comprises an amino acid sequence identical to the amino acid sequence of an endogenous gamma chain-independent Ig-LC receptor encoded by said isolated cell's genome.

6. An isolated and/or recombinant Ig-LC receptor or a functional part, derivative and/or analogue thereof, capable of activating an Ig-LC-dependent signal transduction pathway in a cell, wherein said activation is independent of the presence of a functional gamma chain receptor on said cell.

7. The isolated and/or recombinant Ig-LC receptor of claim 6, wherein said signal transduction pathway comprises an ion channel.

8. The isolated and/or recombinant Ig-LC receptor of claim 6, wherein said isolated and/or recombinant Ig-LC receptor comprises a protein with an apparent molecular weight of between about 37 and about 50 kDa.

9. The isolated and/or recombinant Ig-LC receptor of claim 6, wherein said isolated and/or recombinant Ig-LC receptor comprises a protein with an apparent molecular weight of about 45 kDa.

10. A proteinaceous compound, capable of binding to Ig-LC and a mast cell, said proteinaceous compound comprising an FcRn receptor or a functional part thereof.

11. A proteinaceous compound, capable of binding to Ig-LC and a mast cell, said proteinaceous compound comprising a CD63 receptor or a functional part thereof.

12. An isolated and/or recombinant nucleic acid encoding said isolated and/or recombinant Ig-LC receptor of claim 6.

13. A process of selecting a compound capable of preventing binding of Ig-LC to a receptor; said process comprising:
said isolated and/or recombinant Ig-LC receptor of claim 6, to select a compound capable of preventing binding of Ig-LC to said receptor.

14. A method for at least in part inhibiting Ig-LC-induced signal transduction in a cell, said method comprising:
providing said cell with a compound capable of at least in part inhibiting association of said Ig-LC with an Ig-LC receptor one said cell.

15. The method according to claim 14, wherein said Ig-LC receptor is a receptor for Ig-LC capable of activating an Ig-LC-dependent signal transduction pathway in a cell, independent of the presence of a functional immunoglobulin on said cell.

16. The method according to claim 14, wherein said compound is capable of specifically binding to said receptor for Ig-LC.

17. The method according to claim 15, wherein said compound comprises Ig-LC selected for its capacity not to elicit signal transduction by said Ig-LC receptor.

18. The method according to claim 14, wherein said compound is capable of specifically binding Ig-LC.

19. The method according to claim 18, wherein said compound comprises a peptide comprising an amino acid sequence (AHWSGHCCL (SEQ ID NO: 1)).

20. A method for reducing a hypersensitivity response in an animal, said method comprising:
providing said animal with a compound capable of at least in part inhibiting binding of an Ig-LC to a gamma chain-independent receptor for Ig-LC.

21. A method for determining whether a compound is capable of at least in part inhibiting signal transduction of a gamma chain-independent Ig-LC receptor, said method comprising:
providing a gamma chain receptor-deficient cell comprising said receptor with said compound, and
determining whether Ig-LC-mediated signal transduction is at least in part inhibited in said gamma chain receptor-deficient cell.

22. A compound capable of at least in part inhibiting signal transduction of a gamma chain-independent Ig-LC receptor obtainable by a method according to claim 21.

23. A medicament for treatment of a disease selected from the group consisting of dermatitis, contact dermatitis, asthma, psoriasis, inflammatory bowel disease, rheumatoid arthritis, Sjögrens, systemic lupus erythematosus, multiple sclerosis, and combinations of any thereof, said medicament comprising:
said compound of claim 22 presented in a pharmaceutically acceptable form.

24. The medicament of claim 23, wherein said medicament is formulated and packaged for parenteral or oral administration.

25. A method of treatment of a subject suffering from or at risk of suffering from dermatitis, contact dermatitis, asthma, psoriasis, inflammatory bowel disease, rheumatoid arthritis, Sjögren's systemic lupus erythematosus, and/or multiple sclerosis, said method comprising administering to said subjects said compound of claim 22 with a carrier to a suitable recipient.


SEQUENCE LISTING (3 sequences)

SEQ ID NO: 1 - protein, 9 aa, Artificial Sequence
  a compound capable of at least in part inhibiting an Ig-LC signal transduction pathway
AHWSGHCCL

SEQ ID NO: 2 - DNA, 20 bp, Artificial Sequence
  forward primer
cctgctgggctgtgaactgg

SEQ ID NO: 3 - DNA, 20 bp, Artificial Sequence
  reverse primer
gctccggdgggtagaaggag